(19) United States
(12) Patent Application Publication
(10) Pub. No.: US 2025/0188144 A1
(43) Pub. Date: Jun. 12, 2025

(54) INSULIN FUSION PROTEIN

(71) Applicants: Samantha J. BUSFIELD, Philadelphia, PA (US); Matthew J. WILSON, Philadelphia, PA (US); SCOUT BIO, INC., Philadelphia, PA (US)

(72) Inventors: Samantha J. BUSFIELD, Philadelphia, PA (US); Matthew J. WILSON, Philadelphia, PA (US)

(21) Appl. No.: 18/843,112

(22) PCT Filed: Mar. 1, 2023
(86) PCT No.: PCT/US2023/063510

§ 371 Date: Aug. 30, 2024

Related U.S. Application Data
(60) Provisional application No. 63/315,296, filed on Mar. 1, 2022.

(51) Int. Cl. C07K 14/62; A61K 38/00; C07K 14/765

(52) U.S. Cl. CPC C07K 14/62; C07K 14/765; A61K 38/00; C07K 2319/02; C07K 2319/31

(57) ABSTRACT

Provided are compositions and methods for treating diabetes in a canine or feline. A canine or feline insulin-serum albumin fusion protein is administered to a subject.

### CROSS-REFERENCE TO RELATED APPLICATIONS

This application claims benefit of priority to U.S. Provisional Patent Application No. 63/315,296, filed Mar. 1, 2022, the disclosure of which is incorporated herein by reference in its entirety for all purposes.

### SEQUENCE LISTING

The Sequence Listing XML associated with this application is provided in XML file format and is hereby incorporated by reference into the specification. The name of the XML file containing the Sequence Listing XML is SCTB_017_01WO_ST26.xml. The XML file is 40,960 bytes, and created on Feb. 27, 2023, and is being submitted electronically via USPTO Patent Center.

### FIELD

The disclosure relates generally to compositions and methods for treating diabetes.

### BACKGROUND

Diabetes mellitus is a syndrome associated with protracted hyperglycemia due to loss or dysfunction of insulin secretion by pancreatic beta cells, diminished insulin sensitivity in tissues, or both. In the dog, beta cell loss tends to be rapid and progressive, and is usually due to immune-mediated destruction, vacuolar degeneration, or pancreatitis. In the cat, loss or dysfunction of beta cells is the result of insulin resistance, islet amyloidosis, or chronic lymphoplasmacytic pancreatitis. In humans, beta cell loss is caused by an autoimmune response in Type 1 diabetes and diminished insulin sensitivity in Type 2 diabetes.

Insulin is an endogenous peptide hormone produced by beta cells of the pancreatic islets and it is considered to be the main anabolic hormone of the body. Insulin is the mainstay of therapy for diabetes in mammals. The current standard of care is twice daily insulin injections along with frequent medical office visits and disposable diagnostics that are expensive, time consuming and inconvenient.

The present disclosure provides compositions and methods related to an insulin fusion protein to provide sustained half-life of insulin.

### SUMMARY

The present disclosure provides a fusion protein for treatment of companion animals, comprising a proinsulin and serum albumin, wherein the proinsulin is a canine proinsulin or a feline proinsulin.

In some embodiments, the proinsulin is canine proinsulin.

In some embodiments, the canine proinsulin sequence shares at least 80%, at least 85%, at least 90%, at least 95%, at least 99%, or 100% identity SEQ ID NO: 12.

In some embodiments, the proinsulin is feline proinsulin.

In some embodiments, the feline proinsulin sequence shares at least 80%, at least 85%, at least 90%, at least 95%, at least 99%, or 100% identity SEQ ID NO: 15.

In some embodiments, the fusion protein comprises a polypeptide that shares at least 80%, at least 85%, at least 90%, at least 95%, at least 99%, or 100% identity to SEQ ID NO: 1.

In some embodiments, the fusion protein comprises an N-terminal signal peptide.

In some embodiments, the signal peptide is a canine insulin signal peptide.

In some embodiments, the signal peptide comprises the sequence that shares at least 80%, at least 85%, at least 90%, at least 95%, at least 99%, or 100% identity to MALWMRLLPLLALLALWAPAPTRA (SEQ ID NO: 7).

In some embodiments, the canine proinsulin is a canine proinsulin variant having a mutation at one or more cleavage sites compared to a reference polypeptide sequence as set forth in SEQ ID NO: 10.

In some embodiments, the canine proinsulin comprises K53R, R55K, and L86R mutations compared to a reference polypeptide sequence as set forth in SEQ ID NO: 10.

In some embodiments, the canine proinsulin-serum albumin fusion protein comprises a linker that shares at least 80%, at least 85%, at least 90%, at least 95%, at least 99%, or 100% identity to SEQ ID NO: 8.

In some embodiments, the polynucleotide encoding the fusion protein is operatively linked to a promoter.

In some embodiments, the promoter is a cytomegalovirus enhancer/chicken b-actin promoter.

The present disclosure provides pharmaceutical composition suitable for use in treating a metabolic disease in a canine or feline comprising the fusion protein of an embodiment of the present disclosure.

In some embodiments, the fusion proteins and/or pharmaceutical compositions of the present disclosure are used in a method for treating a canine or feline subject having a metabolic disease, optionally diabetes.

In some embodiments, the fusion proteins and/or pharmaceutical compositions of the present disclosure are used in the manufacture of a medicament for treating a canine or feline subject having a metabolic disease, optionally diabetes.

In some embodiments, the fusion protein is formulated at a concentration of at least about 0.01 mg/kg, at least about 0.05 mg/kg, at least about 0.1 mg/kg, at least about 1 mg/kg, at least about 2 mg/kg, at least about 5 mg/kg, at least about 10 mg/kg, or at least about 15 mg/kg of the fusion protein and/or wherein the fusion protein is administered intravenously.

In some embodiments, the fusion protein is administered at a dose of at least about 0.01 mg, at least about 0.05 mg, at least about 0.1 mg, at least about 1 mg, at least about 2 mg, at least about 5 mg, at least about 10 mg, or at least about 15 mg.

In some embodiments, the fusion protein is administered to the canine or feline subject intravenously.

The present disclosure provides a method of treating a canine or feline subject having a metabolic disease, comprising administering to the canine or feline subject an effective amount of the fusion protein of the present disclosure and/or the pharmaceutical composition of the present disclosure.

In some embodiments, the metabolic disease is diabetes.

In some embodiments, the diabetes is Type 1 diabetes.

In some embodiments, the diabetes is Type 2 diabetes.

In some embodiments, the therapeutically effective amount is administered intravenously.

In some embodiments, the therapeutically effective amount is between 0.01 mg/kg to 15 mg/kg of the fusion protein.

In some embodiments, the therapeutically effective amount is between 1 mg/kg to 15 mg/kg of the fusion protein.

In some embodiments, the method decreases fasting blood glucose in the subject by about 10%, about 15%, about 20%, about 25%, about 30%, about 35%, about 40%, about 45%, or about 50%.

The present disclosure provides a polynucleotide encoding a fusion protein for treatment of companion animals, the fusion protein comprising a proinsulin and serum albumin, wherein the proinsulin is a canine proinsulin or a feline proinsulin.

In some embodiments, the canine proinsulin-serum albumin fusion polynucleotide shares at least 80%, at least 85%, at least 90%, at least 95%, at least 99%, or 100% identity to SEQ ID NO: 2.

In some embodiments, the feline proinsulin-serum albumin fusion polynucleotide shares at least 80%, at least 85%, at least 90%, at least 95%, at least 99%, or 100% identity to SEQ ID NO: 19.

### BRIEF DESCRIPTION OF THE DRAWINGS

FIGS. 1A-1C show schematic diagrams of illustrative canine insulin proteins of the present disclosure. All three proteins incorporate a native signal peptide (SP) and modified furin sites. FIG. 1A shows a schematic diagram of an illustrative canine preproinsulin-serum albumin fusion protein (cINS-Alb). The caINS-Alb protein has a glycine/serine linker that links the A-chain of insulin to canine serum albumin. FIG. 1B shows a schematic diagram of an illustrative canine preproinsulin-transferrin fusion protein (cINS-Tf). The caINS-Tf protein has a glycine/serine linker that links the A-chain of insulin to canine transferrin. FIG. 1C shows a schematic diagram of an illustrative canine preproinsulin protein containing the furin site modifications and serves as a control (cINS-2-1).

FIG. 2 shows in vitro insulin bioactivity of caINS-Alb and caINS-Tf compared to a control insulin standard. EC₅₀ values are listed in the table below the graph. Ligand-induced activation of the insulin receptor in response to increasing concentrations of purified cINS-Alb and cINS-Tf. A reference insulin standard was used as a control. Relative potency is represented as Relative Light Units.

### DETAILED DESCRIPTION

As described elsewhere herein, the present disclosure is predicated, at least in part, on the inventors' surprising finding that insulin fusion proteins achieve sustained bioactivity of insulin in canines and felines. Provided are methods of making and using fusion proteins.

An insulin fusion protein engineered to overcome the short half-life of the native hormone by fusion to a protein with longer half-life (e.g., serum albumin) is a therapeutic advancement for the treatment of diabetes. Long-acting insulin fusion protein expression constructs have been developed for use in canine and feline animals. The expression constructs comprise a secretion signal peptide, as well as a fusion domain intended to prolong the time in circulation of the resulting fusion protein.

The expression constructs are administered to subjects in need thereof. Also provided are methods of using these fusion proteins in regimens for treating type 1 diabetes mellitus (T1DM), type 2 diabetes mellitus (T2DM), or metabolic syndrome in a veterinary subject and increasing the half-life of insulin in a subject.

The present disclosure encompasses insulin-albumin fusion proteins comprising a therapeutic protein having insulin activity. The present disclosure also encompasses polynucleotides comprising, or alternatively consisting of, nucleic acid molecules encoding a therapeutic protein having insulin activity fused to albumin or a fragment (portion) or variant of albumin. Albumin may be fused to the N-terminus, the C-terminus, or both termini of the therapeutic protein having insulin activity. In some embodiments, the albumin is fused to the C-terminus of the proinsulin. The present disclosure also encompasses polynucleotides, comprising nucleic acid molecules encoding proteins comprising a therapeutic protein having insulin activity fused to albumin or a fragment (portion) or variant of albumin, that is sufficient to prolong its activity in vivo.

### Leader Sequence

In one embodiment, the insulin protein comprises a leader sequence, which may comprise a secretion signal peptide. As used herein, the term “leader sequence” refers to any N-terminal sequence of a polypeptide. In one embodiment, the canine or feline insulin proteins described herein comprise a leader, or signal sequence, and proinsulin. The leader sequence is, in one embodiment, a native sequence (canine or feline insulin) leader. In another embodiment, the leader sequence is a heterologous sequence, i.e., derived from another protein than canine or feline insulin.

In one embodiment, the leader is a canine IL-2 sequence. In one embodiment, the IL-2 leader comprises a sequence that shares at least 80%, at least 85%, at least 90%, at least 95%, at least 99%, or 100% identity to SEQ ID NO: 21.

SEQ ID NO: 21:
MYKMQLLSCIALTLVLVANS

In another embodiment, the leader is the native canine insulin sequence. In one embodiment, the canine leader comprises a sequence that shares at least 80%, at least 85%, at least 90%, at least 95%, at least 99%, or 100% identity to SEQ ID NO: 7.

SEQ ID NO: 7:
MALWMRLLPLLALLALWAPAPTRA

In one embodiment, the leader sequence is a feline IL-2 sequence. In one embodiment, the IL-2 leader comprises a sequence that shares at least 80%, at least 85%, at least 90%, at least 95%, at least 99%, or 100% identity to SEQ ID NO: 13.

SEQ ID NO: 13:
MYKIQLLSCIALTLILVINS

In another embodiment, the leader is the native feline insulin sequence. In one embodiment, the canine leader comprises a sequence that shares at least 80%, at least 85%, at least 90%, at least 95%, at least 99%, or 100% identity to SEQ ID NO: 9.

SEQ ID NO: 9:
MAPWTRLLPLLALLSLWIPAPTRA

The leader sequence may be derived from the same species for which administration is ultimately intended, i.e., a canine or feline animal. As used herein, the terms “derived” or “derived from” mean the sequence or protein is sourced from a specific subject species or shares the same sequence as a protein or sequence sourced from a specific subject species. For example, a leader sequence which is “derived from” a canine or feline, shares the same sequence (or a variant thereof, as defined herein) as the same leader sequence as expressed in a canine or feline. However, the specified nucleic acid or amino acid need not actually be sourced from a canine or feline. Various techniques are known in the art which are able to produce a desired sequence, including mutagenesis of a similar protein (e.g., a homolog) or artificial production of a nucleic acid or amino acid sequence. The “derived” nucleic acid or amino acid retains the function of the same nucleic acid or amino acid in the species from which it is “derived”, regardless of actual source of the derived sequence.

### Insulin

Insulin is involved in regulation of glucose utilization in the body. The body's inability to synthesize insulin or cells that are resistant to insulin leads to diabetes mellitus which is characterized by chronic hyperglycemia. Preproinsulin is transcribed as a 110 amino acid chain. Removal of the signal peptide from its N-terminus produces proinsulin. Formation of disulfide bonds between the A- & B-chain components, and removal of the intervening C-chain, produces a biologically active insulin molecule comprising 51 amino acids, in size less than half of the original translation product.

As used herein the term “insulin” refers to insulin or a functional fragment thereof, including proinsulin and preproinsulin, and amino-acid sequence variants of insulin or functional fragments thereof. The disclosure provides proteins comprising canine insulin or feline insulin, as well as polynucleotides and expression vectors encoding such proteins. In some embodiments, the insulin protein comprises a polynucleotide sequence encoding a polypeptide comprising (a) a secretion signal peptide and (b) a proinsulin polypeptide. In one embodiment, the protein comprises a canine IL2 signal peptide and canine proinsulin. In another embodiment, the protein comprises a canine insulin signal peptide and canine proinsulin. The amino acid sequence of native canine proinsulin is shown in SEQ ID NO: 10.

In one embodiment, the protein comprises a feline IL2 signal peptide and feline proinsulin. In another embodiment, the protein comprises a feline insulin signal peptide and feline proinsulin. The amino acid sequence of native feline proinsulin is shown in SEQ ID NO: 11.

In some embodiments, canine or feline insulin includes variants which may include up to about 10% variation from an insulin nucleic acid or amino acid sequence described herein or known in the art, which retain the function of the wild-type sequence. As used herein, by “retain function” it is meant that the nucleic acid or amino acid functions in the same way as the wildtype sequence, although not necessarily at the same level of expression or activity. For example, in one embodiment, a functional variant has increased expression or activity as compared to the wildtype sequence. In another embodiment, the functional variant has decreased expression or activity as compared to the wildtype sequence. In one embodiment, the functional variant has 10%, 20%, 30%, 40%, 50%, 60%, 70%, 80%, 90% or greater increase or decrease in expression or activity as compared to the wildtype sequence (SEQ ID NO: 11).

The canine proinsulin sequence, in one embodiment, contains one or more mutations as compared to the native sequence. These mutations are, in some embodiments, in the cleavage sites between the B/C chains and C/A chains. In one embodiment, one or more of the cleavage sites are mutated to incorporate at least one furin cleavage site at existing protease cleavage sites. In one embodiment, the proinsulin sequence has a K53R mutation. In another embodiment, the proinsulin sequence has a R55K mutation. In another embodiment, the proinsulin sequence has a L86R mutation. In another embodiment, the proinsulin sequence has both K53R and R55K mutations. In another embodiment, the proinsulin sequence has both K53R and L86R mutations. In another embodiment, the proinsulin sequence has both R55K and L86R mutations. In another embodiment, the proinsulin sequence has K53R, R55K, and L86R mutations.

In one embodiment, the canine proinsulin sequence is a sequence that shares at least 80%, at least 85%, at least 90%, at least 95%, at least 99%, or 100% identity SEQ ID NO: 12.

In one embodiment, the feline proinsulin sequence is a sequence that shares at least 80%, at least 85%, at least 90%, at least 95%, at least 99%, or 100% identity SEQ ID NO: 15.

When a variant or fragment of the proinsulin sequence is desired, the coding sequences for these peptides may be generated using site-directed mutagenesis of the wild-type nucleic acid sequence. Alternatively, or additionally, web-based or commercially available computer programs, as well as service-based companies may be used to back translate the amino acids sequences to nucleic acid coding sequences, including both RNA and/or cDNA. See, e.g., backtranseq by EMBOSS; Gene Infinity; and/or ExPasy. In one embodiment, the RNA and/or cDNA coding sequences are designed for optimal expression in the subject species for which administration is ultimately intended, i.e., a canine or a feline.

### Fusion Domains

The disclosure provides fusion proteins comprising a fusion domain. By fusing an insulin to a fusion domain with longer half-life, the insulin fusion protein overcomes the short half-life of the native hormone. In some embodiments, the fusion domain comprises either (i) a canine serum albumin or a functional variant thereof, (ii) a canine IgG Fc or a functional variant thereof, or (iii) a canine transferrin or a functional variant thereof. In some embodiments, the fusion domain comprises a canine serum albumin.

In some embodiments, the fusion domain comprises either (i) a feline serum albumin or a functional variant thereof, (ii) a feline IgG Fc or a functional variant thereof, or (iii) a feline transferrin or a functional variant thereof. In some embodiments, the fusion domain comprises a feline serum albumin.

In some embodiments, the fusion domain is a canine serum albumin comprising a sequence that shares at least 80%, at least 85%, at least 90%, at least 95%, at least 99%, or 100% identity to SEQ ID NO: 16.

In some embodiments, the fusion domain is a canine transferrin comprising a sequence that shares at least 80%, at least 85%, at least 90%, at least 95%, at least 99%, or 100% identity to SEQ ID NO: 17.

In some embodiments, the fusion domain is a feline serum albumin comprising a sequence that shares at least 80%, at least 85%, at least 90%, at least 95%, at least 99%, or 100% identity to SEQ ID NO: 18.

### Insulin Fusion Proteins

The disclosure provides fusion proteins comprising one or more copies of proinsulin, as well as polynucleotides and expression vectors encoding such fusion proteins. In some embodiments, the fusion protein comprises a polynucleotide sequence encoding a fusion protein comprising (a) a leader sequence comprising a secretion signal peptide, (b) a proinsulin, and (c) a fusion domain comprising either (i) an IgG Fe or a functional variant thereof, (ii) an albumin or a functional variant thereof, or (iii) a transferrin or a functional variant thereof. In one embodiment, the fusion protein comprises a thrombin leader sequence, a proinsulin, and an IgG Fc or functional variant thereof. In another embodiment, the fusion protein comprises a thrombin leader sequence, a proinsulin, and an albumin or functional variant thereof.

In some embodiments, the fusion protein comprises a canine insulin leader sequence, a canine proinsulin (K53R, R55K and L86R), a glycine/serine linker, and a canine serum albumin. In an embodiment, the fusion protein comprises a sequence that shares at least 80%, at least 85%, at least 90%, at least 95%, at least 99%, or 100% identity to SEQ ID NO: 1.

In some embodiments, the fusion protein comprises a canine insulin leader sequence, a canine proinsulin (K53R, R55K and L86R), a glycine/serine linker, and a canine transferrin. In an embodiment, the fusion protein comprises a sequence that shares at least 80%, at least 85%, at least 90%, at least 95%, at least 99%, or 100% identity to SEQ ID NO: 3.

In some embodiments, the fusion protein comprises a canine insulin leader sequence and a canine proinsulin (K53R, R55K and L86R). In an embodiment, the fusion protein comprises a sequence that shares at least 80%, at least 85%, at least 90%, at least 95%, at least 99%, or 100% identity to SEQ ID NO: 5.

In some embodiments, the fusion protein comprises a feline insulin leader sequence, a feline proinsulin (K53R, R55K and L86R), a glycine/serine linker, and a feline serum albumin. In an embodiment, the fusion protein comprises a sequence that shares at least 80%, at least 85%, at least 90%, at least 95%, at least 99%, or 100% identity to SEQ ID NO: 6.

In one embodiment, the fusion protein comprises an insulin leader sequence, a proinsulin, and an albumin or functional variant thereof. In one embodiment, the fusion protein comprises an insulin leader sequence, a proinsulin, and a transferrin or functional variant thereof.

In one embodiment, the fusion protein comprises an IL2 leader sequence, a proinsulin, and an albumin or functional variant thereof. In one embodiment, the fusion protein comprises an IL2 leader sequence, a proinsulin, and a transferrin or functional variant thereof.

In addition to the leader sequence, proinsulin, and insulin polypeptides provided herein, nucleic acid sequences (used interchangeably with “polynucleotides”) encoding these polypeptides are provided. In one embodiment, a nucleic acid sequence is provided which encodes for the proinsulin-serum albumin fusion polypeptide described herein. In some embodiments, the nucleic acid sequence which encodes the canine proinsulin-serum albumin fusion comprises a sequence that shares at least 80%, at least 85%, at least 90%, at least 95%, at least 99%, or 100% identity to SEQ ID NO: 2.

In some embodiments, the nucleic acid sequence which encodes the canine proinsulin-transferrin fusion comprises a sequence that shares at least 80%, at least 85%, at least 90%, at least 95%, at least 99%, or 100% identity to SEQ ID NO: 4.

In some embodiments, the nucleic acid sequence which encodes the feline proinsulin-serum albumin fusion comprises a sequence that shares at least 80%, at least 85%, at least 90%, at least 95%, at least 99%, or 100% identity to SEQ ID NO: 19.

The in vivo function and stability of the fusion proteins of the present disclosure may be optimized by adding small peptide linkers, e.g., to prevent potentially unwanted domain interactions or for other reasons. Further, a glycine-rich linker may provide some structural flexibility such that the proinsulin portion can interact productively with the insulin receptor on target cells. Thus, the C-terminus of the proinsulin and the N-terminus of the fusion domain of the fusion protein are, in one embodiment, fused via a linker. In some embodiments, the linker includes 1, 2, 3, or n repeats of a G-rich peptide linker having the sequence (GGGGS) n. In one embodiment, the linker includes 1, 1.5, or 2 repeats of a G-rich peptide linker having the sequence GGGGSGGGGSGGGGS (SEQ ID NO: 14). In one embodiment, the linker includes repeats of a G-rich peptide linker having the sequence GGGGSGGGGSGGGS (SEQ ID NO: 8). In one embodiment, the linker includes repeats of a G-rich peptide linker having the sequence GGGGSGGGGS (SEQ ID NO: 20).

In some embodiments, the fusion protein of the present disclosure comprises, in 5′ to 3′:
(a) canine insulin signal peptide;
(b) canine proinsulin (K53R, R55K and L86R);
(c) Gly/Ser linker; and
(d) canine serum albumin.

In some embodiments, the expression cassette comprises a nucleic acid sequence that shares at least 75%, at least 80%, at least 85%, at least 90%, at least 95%, at least 99%, or 100% identity to SEQ ID NO: 2.

In some embodiments, the fusion protein of the present disclosure comprises, in 5′ to 3′:
(a) feline insulin signal peptide;
(b) feline proinsulin (K53R, R55K and L86R);
(c) Gly/Ser linker; and
(d) feline serum albumin

In some embodiments, the expression cassette comprises a nucleic acid sequence that shares at least 75%, at least 80%, at least 85%, at least 90%, at least 95%, at least 99%, or 100% identity to SEQ ID NO: 19.

### Expression Cassette

In some embodiments, the expression cassette refers to a nucleic acid molecule which comprises the proinsulin fusion construct coding sequences, promoter, and may include other regulatory sequences therefor. The expression cassette may be engineered into a genetic element (e.g., a plasmid) for delivery into cells and purification of the fusion protein for therapeutic administration.

The expression cassette typically contains a promoter sequence as part of the expression control sequences. In one embodiment, a constitutive promoter is used. In the plasmids and expression vectors described herein, a CB7 promoter may be used. CB7 is a chicken B-actin promoter with cytomegalovirus enhancer elements. Other promoters, such as viral promoters, constitutive promoters, regulatable promoters (see, e.g., WO 2011/126808 and WO 2013/04943), or a promoter responsive to physiologic cues may be used may be utilized in the expression vectors described herein.

In some embodiments of the plasmids and expression vectors described herein, a CMV enhancer, chicken beta-Actin promoter and rabbit beta-Globin splice acceptor site (CAG) promoter may be used. In some embodiments of the plasmids and expression vectors described herein, an elongation factor-1 alpha (EF1a) promoter may be used.

In addition to a promoter, an expression cassette and/or an expression vector may contain other appropriate transcription initiation, termination, enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. Illustrative examples of suitable poly A sequences include, e.g., rabbit beta globin, SV40, bovine growth hormone (bGH), and TK polyA.

Illustrative examples of suitable enhancers include, e.g., the alpha fetoprotein enhancer, the TTR minimal promoter/enhancer, LSP (TH-binding globulin promoter/alpha1-microglobulin/bikunin enhancer), amongst others. In one embodiment, the polyA is a rabbit globin polyA.

These control sequences are “operably linked” to the proinsulin fusion construct sequences. As used herein, the term “operably linked” refers to both expression control sequences that are contiguous with the gene of interest and expression control sequences that act in trans or at a distance to control the gene of interest.

In one embodiment, an expression cassette is provided which includes a CB7 promoter, chimeric intron, coding sequence for the protein encoded by SEQ ID NO: 2, and a rabbit beta globin poly A.

In one embodiment, an expression cassette is provided which includes a CB7 promoter, chimeric intron, coding sequence for the protein encoded by SEQ ID NO: 4, and a rabbit beta globin poly A.

In one embodiment, an expression cassette is provided which includes a CB7 promoter, chimeric intron, coding sequence for the protein encoded by SEQ ID NO: 19, and a rabbit beta globin poly A.

The methods used to construct any embodiment of this disclosure are known to those with skill in nucleic acid manipulation and include genetic engineering, recombinant engineering, and synthetic techniques.

In some embodiments, the fusion protein of the present disclosure comprises an expression cassette comprising, in 5′ to 3′:
(a) a cytomegalovirus (CMV) enhancer/chicken b-actin (CAG) promoter;
(b) canine insulin signal peptide;
(c) canine proinsulin (K53R, R55K and L86R);
(d) Gly/Ser linker; and
(e) canine serum albumin

In some embodiments, the expression cassette comprises a nucleic acid sequence that shares at least 75%, at least 80%, at least 85%, at least 90%, at least 95%, at least 99%, or 100% identity to SEQ ID NO: 2.

In some embodiments, the fusion protein of the present disclosure comprises an expression cassette comprising, in 5′ to 3′:
(a) a cytomegalovirus (CMV) enhancer/chicken b-actin (CAG) promoter;
(b) feline insulin signal peptide;
(c) feline proinsulin (K53R, R55K and L86R);
(d) Gly/Ser linker; and
(e) feline serum albumin

In some embodiments, the expression cassette comprises a nucleic acid sequence that shares at least 75%, at least 80%, at least 85%, at least 90%, at least 95%, at least 99%, or 100% identity to SEQ ID NO: 19.

In one embodiment, the nucleic acid sequences encoding the proinsulin fusion constructs described herein are engineered into any suitable genetic element, e.g., naked DNA, phage, transposon, cosmid, RNA molecule (e.g., mRNA), episome, recombinant AAV, etc., which transfers the proinsulin fusion sequences carried thereon to a host cell, e.g., for generating nanoparticles carrying DNA or RNA, virions in a packaging host cell and/or for delivery to a host cell in a subject. In one embodiment, the genetic element is a plasmid. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

In some embodiments, the proinsulin fusion constructs described herein may be delivered via virions including, but not limited to, recombinant AAV (rAAV). Such other virions may include any virus suitable for gene therapy may be used, including but not limited to adenovirus; herpes virus; lentivirus; retrovirus; etc. Suitably, where one of these other virions is generated, it is produced as a replication-defective virion.

### Cell Culture

Animal cells, mammalian cells, cultured cells, animal or mammalian host cells, host cells, recombinant cells, recombinant host cells, manufacturing cell line, and the like, are all terms for the cells that can be maintained in cell culture media. Such cells are typically cell lines obtained or derived from mammals and are able to grow and survive when placed in either monolayer culture or suspension culture in medium containing appropriate nutrients and/or growth factors. Growth factors and nutrients that are necessary for the growth and maintenance of particular cell cultures are able to be readily determined empirically by those having skill in the pertinent art, such as is described, for example, by Barnes and Sato, (1980, Cell, 22:649); in Mammalian Cell Culture, Ed. J. P. Mather, Plenum Press, N Y, 1984; and in U.S. Pat. No. 5,721,121.

In addition, cell culture conditions are typically employed and known for batch, fed-batch, or continuous culturing of cells, with attention paid to pH, e.g., about 6.5 to about 7.5; dissolved oxygen (O₂), e.g., between about 5-90% of air saturation and carbon dioxide (CO₂), agitation and humidity, in addition to temperature.

The cell line cells are typically animal or mammalian cells that can express and secrete, or that can be molecularly engineered to express and secrete, large quantities of a particular protein into the culture medium. It will be understood that the protein of interest produced by a host cell can be endogenous or homologous to the host cell. In one embodiment, the protein of interest is produced and secreted by a Chinese hamster ovary (CHO) host cell. In some embodiments, the protein of interest is a proinsulin-serum albumin fusion protein.

Nonlimiting examples of animal or mammalian host cells suitable for harboring, expressing, and producing proteins for subsequent isolation and/or purification include Chinese hamster ovary cells (CHO).

The cells suitable for culturing in the processes of the present disclosure may contain introduced, e.g., via transformation, transfection, infection, or injection, expression vectors (constructs), such as plasmids and the like, that harbor coding sequences, or portions thereof, encoding the proteins for expression and production in the culturing process. Such expression vectors contain the necessary elements for the transcription and translation of the inserted coding sequence.

In the culturing methods encompassed by the present disclosure, the protein produced by the cells is typically collected, recovered, isolated, and/or purified, or substantially purified, as desired, at the end of the total cell culture period using isolation and purification methods as known and practiced in the art. In some embodiments, the protein of interest is secreted from the cultured cells and is isolated from the culture medium or supernatant. In some embodiments, protein can also be recovered from the host cells, e.g., cell lysates, using methods that are known and practiced in the art.

Methods of Treating Subjects with the Disclosed Compositions

Also provided are compositions which include the fusion proteins described herein. The pharmaceutical compositions described herein are designed for delivery to canine or feline subjects in need thereof by any suitable route or a combination of different routes. Direct delivery to the liver (optionally via intravenous, via the hepatic artery, or by transplant), direct delivery to the pancreas, oral, inhalation, intranasal, intratracheal, intraarterial, intraocular, intravenous, intramuscular, subcutaneous, intradermal, and other parental routes of administration. The fusion proteins described herein may be delivered in a single composition or multiple compositions.

In some embodiments, the pharmaceutical compositions described herein are designed for delivery to canine or feline subjects in need thereof by intramuscular administration.

In some embodiments, the pharmaceutical compositions described herein are designed for delivery to canine or feline subjects in need thereof by intravenous administration.

In some embodiments, the pharmaceutical compositions described herein are designed for delivery to canine or feline subjects in need thereof by subcutaneous administration.

In some embodiments, a course of treatment may involve repeat administration of the same fusion proteins. Still other combinations may be selected using the fusion proteins described herein. In some embodiments, the composition described herein may be combined in a regimen involving other diabetic drugs or protein-based therapies (including e.g., insulin analogues, insulin, oral antihyperglycemic drugs, sulfonylureas, biguanides, thiazolidinediones, and alpha-glucosidase inhibitors). In some embodiments, the composition described herein may be combined in a regimen involving lifestyle changes including dietary and exercise regimens.

As used herein the terms “proinsulin construct”, “proinsulin expression construct” and synonyms include the proinsulin sequence as described herein in combination with a leader (whether native or heterologous). The terms “proinsulin construct”, “proinsulin expression construct” and synonyms can be used to refer to the nucleic acid sequences encoding the proinsulin fusion protein or the expression products thereof.

In one aspect, the present disclosure relates to a method of treating a disease or disorder in a subject in need thereof, comprising administering an effective amount of a proinsulin fusion protein, thereby ameliorating and/or treating one or more of the symptoms of type 1 diabetes, type II diabetes or metabolic syndrome.

In some embodiments, the method comprises administering at least about 0.01 mg/kg, at least about 0.05 mg/kg, at least about 0.1 mg/kg, at least about 1 mg/kg, at least about 2 mg/kg, at least about 5 mg/kg, at least about 10 mg/kg, or at least about 15 mg/kg of the fusion protein.

In some embodiments, a unit dose of proinsulin fusion protein comprises at least about 0.01 mg, at least about 0.05 mg, at least about 0.1 mg, at least about 1 mg, at least about 2 mg, at least about 5 mg, at least about 10 mg, or at least about 15 mg.

In some embodiments, the fusion protein is administered intravenously. In some embodiments, the fusion protein is administered intramuscularly. In some embodiments, the fusion protein is administered subcutaneously. In some embodiments, the therapeutically effective amount of proinsulin fusion protein is administered at a dose of at least about 0.01 mg/kg, at least about 0.05 mg/kg, at least about 0.1 mg/kg, at least about 1 mg/kg, at least about 2 mg/kg, at least about 5 mg/kg, at least about 10 mg/kg, or at least about 15 mg/kg.

In some embodiments, the therapeutically effective amount of proinsulin fusion protein is administered at a unit dose of at least about 0.01 mg, at least about 0.05 mg, at least about 0.1 mg, at least about 1 mg, at least about 2 mg, at least about 5 mg, at least about 10 mg, or at least about 15 mg.

In some embodiments, the therapeutically effective amount of proinsulin fusion protein is administered at a unit dose of at most about 100 mg, at most about 90 mg, at most about 80 mg, at most about 70 mg, at most about 60 mg, at most about 50 mg, at most about 40 mg, at most about 30 mg, at most about 20 mg, or at most about 10 mg.

In some embodiments, the method comprises administering the dose of proinsulin fusion protein once per day, once every 2 days, once every 3 days, once every 4 days, once every 7 days, once every 14 days, once every 21 days, or once every 28 days.

In some embodiments, the method comprises administering the dose of proinsulin fusion protein regimen for at least one week, two weeks, or four weeks.

In some embodiments, the method comprises administering the dose of proinsulin fusion protein regimen for at least one month, two months, four months, six months, eight months, ten months, 12 months, 14 months, 16 months, or 18 months.

In some embodiments, the therapeutically effective amount of proinsulin fusion protein regimen is administered for at least one month, two months, three months, four months, five months, six months, seven months, eight months, nine months, ten months, eleven months, one year, eighteen months, two years, three years, four years, five years, six years, seven years, eight years, nine years, or ten years.

In some embodiments, the method comprises administering the dose of proinsulin fusion protein regimen chronically.

The fusion protein compositions can be formulated in dosage units to contain an amount of fusion protein that is in the range of about 0.1 mg to about 15 mg.

In another aspect, a method of sustained bioactivity of a proinsulin fusion protein in a canine or feline subject is provided. The method includes administering a composition as described herein to a subject in need thereof. In one embodiment, the composition includes a fusion protein containing a proinsulin-serum albumin fusion protein, as described herein. In some embodiments, the proinsulin fusion protein described herein results in an extended half-life of insulin as compared to the native peptide. In some embodiments, the fusion protein provided herein results in a half-life of the proinsulin fusion protein in the subject for at least 5 hours, at least 7 hours, at least 10 hours, at least 15 hours, at least 20 hours, at least 25 hours, at least 30 hours, at least 35 hours, or at least 40 hours. In one embodiment, the fusion proteins may be delivered in volumes from 1 μL to about 100 mL for a veterinary subject. See, e.g., Diehl et al, J. Applied Toxicology, 21:15-23 (2001) for a discussion of good practices for administration of substances to various veterinary animals. This document is incorporated herein by reference. As used herein, the term “dosage” can refer to the total dosage delivered to the subject in the course of treatment, or the amount delivered in a single (of multiple) administration.

In one embodiment, the composition is administered in combination with an effective amount of insulin. Various commercially available insulin products are known in the art, including, without limitation, protamine zinc recombinant human insulin (ProZinc®), porcine insulin zinc suspension (Vetsulin®), and insulin glargine (Lantus®). In some embodiments, combination of the fusion proteins described herein with insulin decreases insulin dose requirements in the subject, as compared to prior to treatment with the fusion proteins. Such dose requirements may be reduced by 10% or more, 20% or more, 30% or more, 40% or more, 50% or more, 60% or more, 70% or more, 80% or more, or 90% or more. The treating physician may determine the correct dosage of insulin needed by the subject. For example, the subject may be being treated using insulin or other therapy, which the treating physician may continue upon administration of the fusion proteins. Such insulin or other co-therapy may be continued, reduced, or discontinued as needed subsequently.

In one embodiment, the subject is delivered a therapeutically effective amount of a composition described herein. As used herein, a “therapeutically effective amount” refers to the amount of the fusion protein that delivers in the target cells an amount of proinsulin-serum albumin sufficient to reach the therapeutic goal. In certain embodiments, the therapeutic goal is to ameliorate or treat one or more of the symptoms of type I diabetes, type II diabetes or metabolic syndrome. A therapeutically effective amount may be determined based on an animal model, rather than a canine or feline subject. In another embodiment, the therapeutic goal is remission of the metabolic disease in the subject.

The above-described fusion proteins may be delivered to host cells according to published methods. The fusion proteins, preferably suspended in a physiologically compatible carrier, may be administered to a desired subject including a canine. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

In some embodiments, the compositions of the disclosure may contain, in addition to the fusion proteins and/or variants and carrier(s), other conventional pharmaceutical ingredients, such as preservatives, or chemical stabilizers. Illustrative preservatives include chlorobutanol, potassium sorbate, sorbic acid, sulfur dioxide, propyl gallate, the parabens, ethyl vanillin, glycerin, phenol, and parachlorophenol. Illustrative chemical stabilizers include gelatin and albumin.

In some embodiments, the fusion proteins of the present disclosure are formulated as a depot injection. A depot injection formulation delivers a fusion protein (e.g., a proinsulin-serum albumin fusion protein) at a tunable, predetermined rate within the therapeutic range for a specified period. Release can take place directly at the site of action for a local treatment or at a systemic level, thus reducing the adverse side effects of the fusion protein to a minimum.

In some embodiments, the proinsulin-serum albumin fusion protein of the present disclosure is formulated as a depot injection.

The recombinant fusion proteins described herein may be used in preparing a medicament for delivering a proinsulin fusion protein to a subject in need thereof, supplying insulin having an increased half-life to a subject, and/or for treating type I diabetes, type II diabetes, or metabolic syndrome in a subject.

In one aspect, a method of treating diabetes is provided. The method includes administering a composition as described herein to a canine or feline subject in need thereof. In one embodiment, the composition includes a proinsulin fusion, as described herein.

In another embodiment, a method for treating type 2 diabetes in a canine or feline is provided. The method includes administering a proinsulin fusion protein as described herein.

In another embodiment, a method for treating type 1 diabetes in a canine or feline is provided. The method includes administering a proinsulin fusion protein as described herein.

In another aspect, a method of treating a metabolic disease in a canine or feline is provided. The method includes administering a composition as described herein to a canine or feline subject in need thereof. In one embodiment, the composition includes a proinsulin fusion protein, as described herein. In one embodiment, the metabolic disease is Type I diabetes. In one embodiment, the metabolic disease is Type II diabetes. In one embodiment, the metabolic disease is metabolic syndrome.

In another embodiment, a method for treating diabetes in a canine or feline is provided. The method includes administering a proinsulin-serum albumin fusion protein as described herein, wherein the fusion protein is administered after insulin is administered in a subject.

In another embodiment, a method for preventing cataract formation in diabetic canines or felines is provided. The method includes administering a proinsulin-serum albumin fusion protein as described herein.

In another embodiment, a method for reducing the blood glucose concentration in diabetic canines or felines is provided. The method includes administering a proinsulin-serum albumin fusion protein as described herein.

As used herein, the term “treatment” or “treating” is defined encompassing administering to a subject one or more compounds or compositions described herein for the purposes of amelioration of one or more symptoms of type I diabetes, type II diabetes (T2DM) or metabolic syndrome. “Treatment” can thus include one or more of reducing progression of type I diabetes, type II diabetes or metabolic syndrome, reducing the severity of the symptoms, removing the disease symptoms, delaying progression of disease, or increasing efficacy of therapy in a given subject.

As used herein, the term “remission” refers to the ability to cease insulin treatment when the cat or dog no longer exhibits clinical signs of diabetes and has normal blood glucose levels.

In another embodiment, a method for treating T2DM in a feline or canine is provided. The method includes administering a fusion protein as described herein.

In another aspect, a method of treating a metabolic disease in a feline or canine is provided. The method includes administering a composition as described herein to a feline or canine subject in need thereof. In one embodiment, the composition includes a proinsulin fusion protein, as described herein.

In another aspect, a method of reducing fasting blood sugar in a canine or feline subject is provided. The method includes administering a composition as described herein to a subject in need thereof. In one embodiment, the composition includes a proinsulin-serum albumin fusion protein, as described herein. In some embodiments, the method provided herein decreases fasting blood glucose in the subject by at least about 10%, at least about 15%, at least about 20%, at least about 25%, at least about 30%, at least about 35%, at least about 40%, at least about 45%, or at least about 50%. In some embodiments, the method provided herein decreases fasting blood glucose in the subject by about 20%. In some embodiments, the method provided herein decreases fasting blood glucose in the subject by about 30%. In some embodiments, the method provided herein decreases fasting blood glucose in the subject by about 40%.

### Definitions

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description is for the purpose of describing particular embodiments only and is not intended to be limiting. All publications, patent applications, patents and other references mentioned herein are incorporated by reference in their entirety. In case of a conflict in terminology, the present specification is controlling.

A reference to “one embodiment” or “another embodiment” in describing an embodiment does not imply that the referenced embodiment is mutually exclusive with another embodiment (e.g., an embodiment described before the referenced embodiment), unless expressly specified otherwise.

The terms “identical” or percent “identity,” in the context of two or more nucleic acids or polypeptide sequences, refer to two or more sequences or subsequences that are the same or have a specified percentage of amino acid residues or nucleotides that are the same; i.e., share at least about 80% identity, for example, at least about 85%, 90%, 91%, 92%, 93%, 94%, 95%, 96%, 97%, 98%, 99% identity over a specified region to a reference sequence, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. In some embodiments, the identity exists over a region that is at least about 25 amino acids or nucleotides in length, for example, over a region that is 50, 100, 200, 300, 400 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. In some embodiments, BLAST and BLAST 2.0 algorithms and the default parameters are used.

The terms “percent (%) identity”, “sequence identity”, “percent sequence identity”, or “percent identical” in the context of amino acid sequences refers to the residues in the two sequences which are the same when aligned for correspondence. Percent identity may be readily determined for amino acid sequences over the full-length of a protein, polypeptide, about 70 amino acids to about 100 amino acids, or a peptide fragment thereof or the corresponding nucleic acid sequence coding sequences. A suitable amino acid fragment may be at least about 8 amino acids in length and may be up to about 150 amino acids. Generally, when referring to “identity”, “homology”, or “similarity” between two different sequences, “identity”, “homology” or “similarity” is determined in reference to “aligned” sequences.

“Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs.

The term “amino acid substitution” and its synonyms are intended to encompass modification of an amino acid sequence by replacement of an amino acid with another, substituting, amino acid. The substitution may be a conservative substitution. It may also be a non-conservative substitution. The term conservative, in referring to two amino acids, is intended to mean that the amino acids share a common property recognized by one of skill in the art. For example, amino acids having hydrophobic nonacidic side chains, amino acids having hydrophobic acidic side chains, amino acids having hydrophilic nonacidic side chains, amino acids having hydrophilic acidic side chains, and amino acids having hydrophilic basic side chains. Common properties may also be amino acids having hydrophobic side chains, amino acids having aliphatic hydrophobic side chains, amino acids having aromatic hydrophobic side chains, amino acids with polar neutral side chains, amino acids with electrically charged side chains, amino acids with electrically charged acidic side chains, and amino acids with electrically charged basic side chains. Both naturally occurring and non-naturally occurring amino acids are known in the art and may be used as substituting amino acids in embodiments. Methods for replacing an amino acid are well known to the skilled in the art and include, but are not limited to, mutations of the nucleotide sequence encoding the amino acid sequence. Reference to “one or more” herein is intended to encompass the individual embodiments of, for example, 1, 2, 3, 4, 5, 6, or more.

The coding sequences may be designed for optimal expression using codon optimization. Codon-optimized coding regions can be designed by various different methods. This optimization may be performed using methods which are available on-line, published methods, or a company which provides codon optimizing services. One codon optimizing method is described, e.g., in International Patent Application Pub. No. WO 2015/012924, which is incorporated by reference herein. Briefly, the nucleic acid sequence encoding the product is modified with synonymous codon sequences. Suitably, the entire length of the open reading frame (ORF) for the product is modified. However, in some embodiments, only a fragment of the ORF may be altered. By using one of these methods, one can apply the frequencies to any given polypeptide sequence and produce a nucleic acid fragment of a codon-optimized coding region which encodes the polypeptide.

In an embodiment, the insulin-serum albumin fusion is humanized, caninized, felinized or equinized.

By “humanized” is meant that the fusion protein comprises an amino acid sequence that is compatible with humans, such that the amino acid sequence is unlikely to be seen as foreign by the immune system of a human subject.

By “caninized” is meant that the fusion protein comprises an amino acid sequence that is compatible with canine, such that the amino acid sequence is unlikely to be seen as foreign by the immune system of a canine subject. In the present disclosure, the term for a polypeptide preceded by the prefix “ca” refers to a variant of the human polypeptide in which the human fusion domain is replaced with the canine homolog of that fusion domain and, where the proinsulin is a fragment or variant of a human protein, the proinsulin is replaced with the canine homolog of that fragment or variant.

By “felinized” is meant that the fusion protein comprises an amino acid sequence that is compatible with feline, such that the amino acid sequence is unlikely to be seen as foreign by the immune system of a feline subject. In the present disclosure, the term for a polypeptide preceded by the prefix “fe” refers to a variant of the human polypeptide in which the human fusion domain is replaced with the feline homolog of that fusion domain and, where the proinsulin is a fragment or variant of a human protein, the proinsulin is replaced with the feline homolog of that fragment or variant.

By “equinized” is meant that the fusion protein comprises an amino acid sequence that is compatible with equine, such that the amino acid sequence is unlikely to be seen as foreign by the immune system of an equine subject.

As noted elsewhere herein, the present disclosure extends to fusion proteins that are compatible with species other than human, canine, feline, and equine. In this context, the fusion proteins can be referred to as “speciesized”, referring to the target species to which the molecule will be administered.

In some embodiments, the compositions and methods described herein are intended to be for use in feline animals. The term feline (family Felidae) refers to any of 37 cat species that among others include the cheetah, puma, jaguar, leopard, lion, lynx, tiger, and domestic cat. In an embodiment, the subject is a domestic cat. In some embodiments, the compositions and methods described herein are intended to be for use in canine animals. The term canine refers to any of species found in the Canidae family that among others includes domestic dogs, wolves, and foxes. In an embodiment, the subject is a domestic dog, also known as Canis lupus familiaris or Canis familiaris.

As used in the description of the disclosure and the appended claims, the singular forms “a”, “an”, and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (or).

As used herein, the phrase “consisting essentially of” refers to the genera or species of active pharmaceutical agents recited in a method or composition, and further can include other agents that, on their own do not have substantial activity for the recited indication or purpose.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% from the reference given, unless otherwise specified.

The term “regulation” or variations thereof as used herein refers to the ability of a composition to inhibit one or more components of a biological pathway.

As used herein, “disease”, “disorder” and “condition” are used interchangeably, to indicate an abnormal state in a subject.

The terms “subject,” “individual,” and “patient” interchangeably refer to a mammal, a human or a non-human primate, domesticated mammals (e.g., canine or feline), laboratory mammals, and agricultural mammals. In various embodiments, the subject can be a human (e.g., adult male, adult female, adolescent male, adolescent female, male child, female child). In various embodiments, the subject is a companion animal. Illustrative companion animals include, but are not limited to, dogs, cats, horses, rabbits, ferrets, birds, and guinea pigs. In various embodiments, the subject is a canine. In various embodiments, the subject is a feline. In various embodiments, the subject is a mammal.

As used herein, the term “target cell” refers to any target cell in which expression of a protein is desired. In certain embodiments, the target cell is a liver cell. In some embodiments, the target cell is a muscle cell.

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences (also referred to as elements) that are contiguous or non-contiguous with the nucleic acid sequence and regulatory sequences that act in trans or cis nucleic acid sequence. Such regulatory sequences typically include, e.g., one or more of a promoter, an enhancer, a transcription factor, transcription terminator, an intron, sequences that enhance translation efficiency (i.e., a Kozak consensus sequence), efficient RNA processing signals such as slicing and a polyadenylation sequence, sequences that stabilize cytoplasmic mRNA, for example Woodchuck Hepatitis Virus (WHP) posttranslational Regulatory Element (WPRE), and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequence, i.e., 5′-untranslated regions (5′ UTR). In certain embodiments, the expression cassette comprises nucleic acid sequence of one or more of gene products. In some embodiments, the expression cassette can be a monocistronic or a bicistronic expression cassette.

As used herein, “administering” refers to local and systemic administration, e.g., including enteral, parenteral, pulmonary, and topical/transdermal administration. Routes of administration for pharmaceutical ingredients that find use in the methods described herein include, e.g., oral (per os (P.O.)) administration, nasal or inhalation administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, intralesional administration, or subcutaneous (“sc”) administration, or the implantation of a slow-release device e.g., a mini-osmotic pump, a depot formulation, etc., to a subject. Parenteral administration includes, e.g., intravenous, intramuscular, intraarterial, intrarenal, intraurethral, intracardiac, intracoronary, intramyocardial, intradermal, epidural, subcutaneous, intraperitoneal, intraventricular, ionophoretic and intracranial.

The terms “systemic administration” and “systemically administered” refer to a method of administering a pharmaceutical ingredient or composition to a mammal so that the pharmaceutical ingredient or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

The term “effective amount” or “pharmaceutically effective amount” refer to the amount and/or dosage, and/or dosage regime of one or more pharmaceutical ingredients (e.g., fusion proteins) necessary to bring about the desired result.

As used herein, the terms “treating” and “treatment” refer to delaying the onset of, retarding or reversing the progress of, reducing the severity of, or alleviating or preventing either the disease or condition to which the term applies, or one or more symptoms of such disease or condition. The terms “treating” and “treatment” also include preventing, mitigating, ameliorating, reducing, inhibiting, eliminating and/or reversing one or more symptoms of the disease or condition.

The term “mitigating” refers to reduction or elimination of one or more symptoms of that pathology or disease, and/or a reduction in the rate or delay of onset or severity of one or more symptoms of that pathology or disease, and/or the prevention of that pathology or disease. In some embodiments, the reduction or elimination of one or more symptoms of pathology or disease can include, e.g., measurable and sustained decrease of fasting blood glucose.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

The section headings used herein are for organizational purposes only and are not to be construed as limiting the subject matter described.

While illustrative embodiments have been illustrated and described, it will be appreciated that various changes can be made therein without departing from the spirit and scope of the disclosure.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what is regarding as the disclosure.

### Example 1: Generation of a Half-Life Extended Canine Insulin-Serum Albumin Fusion and Evaluation of In Vitro Potency of Canine Insulin Fusion Proteins

A canine insulin-serum albumin fusion protein has been developed for the management of hyperglycemia and hyperglycemia-associated clinical signs of diabetes mellitus in dogs.

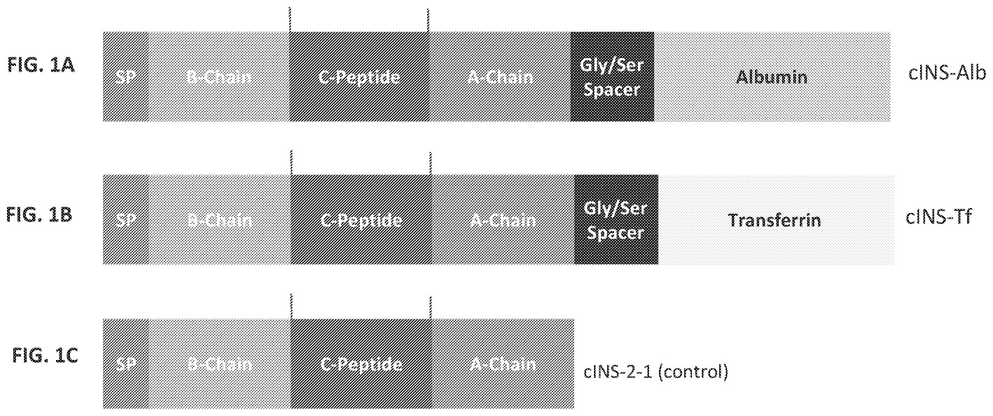

A canine preproinsulin-serum albumin fusion protein (cINS-Alb) was generated by constructing a fusion polypeptide containing the following elements (FIG. 1A) (SEQ ID NO: 1):
Native canine insulin signal peptide (SP)
Canine proinsulin, where the native sequence has been modified at 3 amino acid positions (K53R, R55K and L86R) to incorporate 2 furin cleavage sites at existing protease cleavage sites (B-Chain, C-Peptide, A-Chain)
Gly/Ser linker comprising the sequence GGGGSGGGGSGGGS (SEQ ID NO: 8)
Canine serum albumin

A canine preproinsulin fusion protein was made with the same elements as above except the canine serum albumin sequence was replaced with canine transferrin (cINS-Tf) (FIG. 1B) (SEQ ID NO: 3). A control canine preproinsulin sequence, with the 3 amino acid modifications (K53R, R55K and L86R) was also generated as a control (cINS-2-1) (FIG. 1C) (SEQ ID NO: 5).

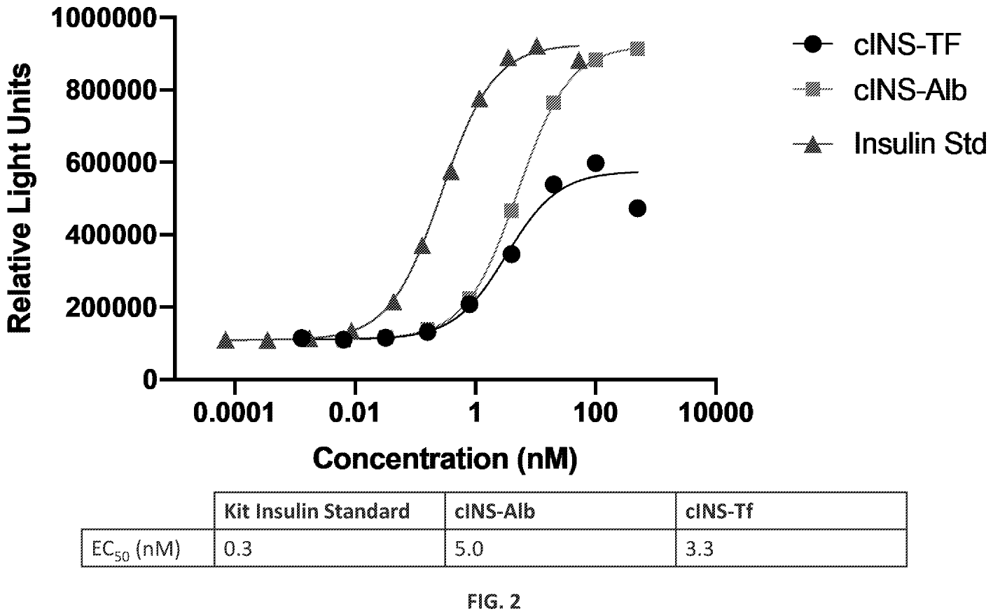

A study was conducted to evaluate the in vitro potency of the two fusion proteins (cINS-Alb and cINS-Tf), C-terminal histidine-tagged versions of each of the proteins was produced in mammalian cells following transient transfection of expression plasmids containing the cDNA encoding the respective proteins. The proteins were purified from the cell supernatant via nickel affinity chromatography and were assayed for insulin bioactivity using a PathHunterÒ™ Insulin Bioassay kit (Eurofins™ DiscoverX Products, LLC) as per the kit instructions. A standard insulin provided in the kit was used as a reference control. Both cINS-Alb and cINS-Tf demonstrated bioactivity in this assay (FIG. 2). The modifications made to the insulin molecule in both of the fusion proteins resulted in a small loss in potency as indicated by the shift in EC₅₀ values.

1. A fusion protein for treatment of companion animals, comprising a proinsulin and serum albumin, wherein the proinsulin is a canine proinsulin or a feline proinsulin.

2. The fusion protein of claim 1, wherein proinsulin is canine proinsulin.

3. The fusion protein of claim 2, wherein the canine pro insulin sequence shares at least 80% identity SEQ ID NO: 12.

4. The fusion protein of claim 1, wherein proinsulin is feline proinsulin.

5. The fusion protein of claim 4, wherein feline proinsulin sequence shares at least 80% identity SEQ ID NO: 15.

6. The fusion protein of claim 1, wherein the fusion protein comprises a polypeptide that shares at least 80% identity to SEQ ID NO: 1.

7. The fusion protein of claim 1, wherein the fusion protein comprises an N-terminal signal peptide.

8. The fusion protein of claim 7, wherein the signal peptide is a canine insulin signal peptide.

9. The fusion protein of claim 7, wherein the signal peptide comprises the sequence that shares at least 80% identity to MALWMRLLPLLALLALWAPAPTRA (SEQ ID NO: 7).

10. The fusion protein of claim 1, wherein the canine proinsulin is a canine proinsulin variant having a mutation at one or more cleavage sites compared to a reference polypeptide sequence as set forth in SEQ ID NO: 10.

11. The fusion protein of claim 1, wherein the canine proinsulin comprises K53R, R55K, and L86R mutations compared to a reference polypeptide sequence as set forth in SEQ ID NO: 10.

12. The fusion protein of claim 1, wherein the canine proinsulin-serum albumin fusion protein comprises a linker that shares at least 80% identity to SEQ ID NO: 8.

13. The fusion protein of claim 1, wherein the polynucleotide encoding the fusion protein is operatively linked to a promoter.

14. The fusion protein of claim 13, wherein the promoter is a cytomegalovirus enhancer/chicken b-actin promoter.

15. A pharmaceutical composition suitable for use in treating a metabolic disease in a canine or feline comprising the fusion protein of claim 1.

16. The fusion protein of claim 1 for use in a method for treating a canine or feline subject having a metabolic disease.

17. Use of the fusion protein of claim 1 in the manufacture of a medicament for treating a canine or feline subject having a metabolic disease.

18. The fusion protein of claim 1, wherein the fusion protein is formulated to be administered at a concentration of at least about 0.01 mg/kg of the fusion protein and/or wherein the fusion protein is administered intravenously.

19. The fusion protein of claim 1, wherein the fusion protein is administered at a dose of at least about 0.01 mg.

20. The fusion protein of claim 1, wherein the fusion protein is administered to the canine or feline subject intravenously.

21. A method of treating a canine or feline subject having a metabolic disease, comprising administering to the canine or feline subject an effective amount of the fusion protein of claim 1.

22. The method of claim 21, wherein the metabolic disease is diabetes.

23. The method of claim 22, wherein the diabetes is Type 1 diabetes.

24. The method of claim 22, wherein the diabetes is Type 2 diabetes.

25. The method of claim 21, wherein the effective amount is administered intravenously.

26. The method of claim 21, wherein the effective amount is between 0.01 mg/kg to 15 mg/kg of the fusion protein.

27. The method of claim 21, wherein the effective amount is between 1 mg/kg to 15 mg/kg of the fusion protein.

28. The method of claim 21, wherein the method decreases fasting blood glucose in the subject by at least about 10%.

29. A polynucleotide encoding a fusion protein for treatment of companion animals, the fusion protein comprising a proinsulin and serum albumin, wherein the proinsulin is a canine proinsulin or a feline proinsulin.

30. The polynucleotide of claim 29, wherein the canine proinsulin-serum albumin fusion polynucleotide shares at least 80% identity to SEQ ID NO: 2.

31. The polynucleotide of claim 29, wherein the feline proinsulin-serum albumin fusion polynucleotide shares at least 80% identity to SEQ ID NO: 19.


SEQUENCE LISTING (21 sequences)

SEQ ID NO: 1 - protein, 708 aa, synthetic construct
  REGION 1..708: note = cINS-Alb
MALWMRLLPLLALLALWAPAPTRAFVNQHLCGSHLVEALYLVCGERGFFYTPRAKREVEDLQVRDVELAGAPGEGGLQPLALEGARQKRGIVEQCCTSICSLYQLENYCNGGGGSGGGGSGGGSEAYKSEIAHRYNDLGEEHFRGLVLVAFSQYLQQCPFEDHVKLAKEVTEFAKACAAEESGANCDKSLHTLFGDKLCTVASLRDKYGDMADCCEKQEPDRNECFLAHKDDNPGFPPLVAPEPDALCAAFQDNEQLFLGKYLYEIARRHPYFYAPELLYYAQQYKGVFAECCQAADKAACLGPKIEALREKVLLSSAKERFKCASLQKFGDRAFKAWSVARLSQRFPKADFAEISKVVTDLTKVHKECCHGDLLECADDRADLAKYMCENQDSISTKLKECCDKPVLEKSQCLAEVERDELPGDLPSLAADFVEDKEVCKNYQEAKDVFLGTFLYEYSRRHPEYSVSLLLRLAKEYEATLEKCCATDDPPTCYAKVLDEFKPLVDEPQNLVKTNCELFEKLGEYGFQNALLVRYTKKAPQVSTPTLVEVSRKLGKVGTKCCKKPESERMSCADDFLSVVLNRLCVLHEKTPVSERVTKCCSESLVNRRPCFSGLEVDETYVPKEFNAETFTFHADLCTLPEAEKQVKKQTALVELLKHKPKATDEQLKTVMGDFGAFVEKCCAAENKEGCFSEEGPKLVAAAQAALV

SEQ ID NO: 2 - DNA, 2127 bp, synthetic construct
  misc_feature 1..2127: note = cINS-Alb
  source 1..2127: mol_type = other DNA
atggctctgtggatgagactgctgccactgctggctctgctcgcactgtgggctccagctcctacaagagccttcgtgaaccagcacctgtgcggctctcatctggtggaagccctgtatctcgtgtgcggcgagaggggctttttctacacccctagggccaagagagaggtcgaggatctgcaagtgcgcgacgttgaactggctggtgctcctggcgaaggtggactgcaacctctggctctggaaggcgccagacagaaaaggggcatcgtggaacagtgctgcaccagcatctgcagcctgtaccagctggaaaactactgcaatggcggcggaggatctggcggaggcggtagtggtggtggatctgaggcctacaagtccgagatcgcccaccggtacaatgacctgggcgaagaacacttcagaggcctggtgctggtggccttcagccagtacctgcagcagtgccctttcgaggaccacgtgaagctggccaaagaagtgaccgagttcgccaaagcctgtgccgccgaagaaagcggcgccaattgcgacaaaagcctgcacaccctgttcggcgacaagctgtgtacagtggccagcctgagggataagtacggcgacatggccgactgctgcgagaagcaagagcccgacagaaacgagtgctttctggcccacaaggacgacaaccctggcttccctcctctggtggctcctgaacctgatgctctgtgtgccgcctttcaggacaacgagcagctgttcctgggcaagtacctgtacgaaatcgccaggcggcacccctacttttacgcccctgaactgctgtactacgcccagcagtacaagggcgtgttcgccgaatgttgtcaggccgccgataaggccgcttgtctgggaccaaaaatcgaggccctgcgcgagaaggtgctgctgtctagcgccaaagaaaggttcaagtgcgcctctctgcagaagttcggcgatagggccttcaaggcttggagcgtggcaaggctgagccagagattccctaaggccgacttcgccgagatcagcaaggtggtcaccgacctgaccaaggtgcacaaagagtgctgccacggcgacctgctggaatgcgctgatgatagagccgatctggccaagtacatgtgcgagaaccaggacagcatcagcaccaagctgaaagagtgttgcgacaagcccgtgctggaaaagtctcagtgtctggccgaggtggaaagggatgagctgcctggggatctgccttctctggccgctgacttcgtcgaggacaaagaagtctgcaagaactaccaagaggccaaggatgtgtttctgggcaccttcctgtacgagtactccagaaggcaccccgagtacagcgttagcctgctgctgaggctggctaaagagtacgaggccacactggaaaaatgctgcgccaccgacgatcctccaacctgctatgccaaggtgctggacgagttcaagcccctggtggacgagcctcagaacctggtcaagaccaactgcgagctgttcgagaagctgggagagtacggcttccagaacgccctgctcgtgcggtacacaaaaaaggcccctcaggtgtccacacctacactggtcgaggtgtcccggaaactgggaaaagtgggcaccaagtgttgcaagaagcctgagagcgagaggatgtcctgcgccgacgatttcctgagcgtggtgctgaacagactgtgcgtgctgcacgagaaaacccctgtgtctgagcgcgtgaccaagtgctgctctgagagcctggtcaacagacggccttgtttcagcggcctggaagtggacgaaacctacgtgcccaaagagttcaacgccgaaaccttcaccttccacgccgacctgtgtaccctgcctgaggccgagaaacaagtgaagaaacagacagccctggtcgagctgctgaagcacaagcctaaagccaccgatgagcagctgaaaaccgtgatgggagacttcggcgccttcgtggaaaagtgttgtgccgcagagaacaaagagggatgcttcagcgaagagggccccaagctggttgctgctgctcaagctgccctggtgtaa

SEQ ID NO: 3 - protein, 809 aa, synthetic construct
  REGION 1..809: note = cINS-Tf
MALWMRLLPLLALLALWAPAPTRAFVNQHLCGSHLVEALYLVCGERGFFYTPRAKREVEDLQVRDVELAGAPGEGGLQPLALEGARQKRGIVEQCCTSICSLYQLENYCNGGGGSGGGGSGGGSVSPEKTVRWCTVSNHEASKCSSFMENMKTVLENGPFVSCVKRTSYLECIKAIWANEADAVTLDAGLVFEAGLNPYNLKPVVAEFYGKDKQTRYYAVAVVKKSSTFNLNQLQGKKSCHTGLGRSAGWNIPMGLLYWKLPEPRESLQKAASSFFTASCVPCVDRTAFPKLCQLCVGKGTDKCACSNHEPYFGYSGAFKCLMEDAGEVAFVKHSTVLENLPNKADLDQYELLCPDNKRKPVGEYKQCYLAKVPSHAVVARSVGGKEDLIWELLNQAQEHYGKDKSKVFQLFSSTLGKDLLFKDSAEGFFRIPPKMDTWLYLGYEYVTALRNLREDVRPDTPRDECKKVKWCAVGHHEIAKCDEWSVNSEGKIECESAESTEDCIAKIAKGEADAMSLDGGYIYIAGQCGLVPVLAENYKTQGSTCSNTAEEGYLAVAVVKRLDKTISWNNLQGRKSCHTAVDRTAGWNIPMGLLYNRINHCEDKFFSQGCAPGSMRNSSLCALCIGSANVPGKECVPNNHERYYGYTGAFRCLVEKGDVAFVKDQTVLQNTGGKNTEDWAKDLKEEDFELLCPDGQRKSVDKAPECFLAKAPNHAVVSRKDKASCVSKMLLDQQLLFGRNGNDCSGKFCLFHSATKDLLFRDDTQCLAKLPEDTTYKSYLGAEYITAVANLRQCSTSKLLEACTFHTS

SEQ ID NO: 4 - DNA, 2430 bp, synthetic construct
  misc_feature 1..2430: note = cINS-Tf
  source 1..2430: mol_type = other DNA
atggctctgtggatgagactgctgccactgctggctctgctcgcactgtgggctccagctcctacaagagccttcgtgaaccagcacctgtgcggctctcatctggtggaagccctgtatctcgtgtgcggcgagaggggctttttctacacccctagggccaagagagaggtcgaggatctgcaagtgcgcgacgttgaactggctggtgctcctggcgaaggtggactgcaacctctggctctggaaggcgccagacagaaaaggggcatcgtggaacagtgctgcaccagcatctgcagcctgtaccagctggaaaactactgcaatggcggcggaggatctggcggaggcggttcaggtggtggatctgtgtctcccgaaaagaccgtgcggtggtgcaccgtgtctaatcacgaggccagcaagtgcagctccttcatggaaaacatgaagaccgtcctggaaaacggccccttcgtgtcctgcgtgaagaggaccagctacctggaatgcatcaaggccatctgggccaacgaggccgatgccgttacactggatgccggactggtgtttgaggccggactgaacccctacaacctgaagcctgtggtggccgagttctacggcaaggataagcagacccggtactacgccgtggccgtggtcaagaagtccagcaccttcaacctgaatcagctgcagggcaagaagtcttgccacaccggactgggaagaagcgccggctggaatatccctatgggcctgctgtactggaagctgcccgagcctagagagtctctgcagaaggccgccagcagcttcttcaccgctagctgtgtgccttgcgtggacaggaccgcctttcctaagctgtgccagctgtgtgtcggcaagggcaccgataagtgcgcctgcagcaatcacgagccctacttcggatacagcggcgccttcaagtgcctgatggaagatgctggcgaggtggcctttgtgaagcactccacagtgctggaaaatctgcccaacaaggccgacctggaccagtacgaactgctgtgccccgacaacaagagaaagcccgtgggcgagtacaagcagtgctacctggccaaagtgcctagccatgccgtggtggctagaagcgtcggcggaaaagaggacctgatctgggagctgctgaatcaggcccaagagcactacggaaaggacaagagcaaggtgttccagctgttcagcagcaccctgggcaaagacctgctgttcaaggatagcgccgagggcttcttcaggatccctcctaagatggacacctggctgtacctgggctacgagtacgtgacagccctgaggaacctgcgggaagatgtgcggcctgacacacccagggacgagtgcaagaaagtgaagtggtgcgccgtgggccaccacgagatcgccaaatgtgatgagtggagcgtgaacagcgagggcaagatcgagtgcgagtctgccgagtctaccgaggactgcattgccaaaatcgccaaaggcgaggccgacgccatgtctctggatggcggctacatctatatcgccggccagtgtggactggtgcctgtgctggctgagaactacaagacccagggctccacctgtagcaacaccgccgaagagggatatctggccgtggctgtcgtgaagagactggacaagaccatctcctggaacaacctgcaggggcgcaagagctgtcacaccgccgttgatagaaccgccggatggaacattccaatgggactcctctacaaccggatcaaccactgcgaggacaagttctttagccagggatgtgcccctggcagcatgaggaatagtagcctgtgcgccctgtgcatcggcagcgctaatgtgcctggcaaagaatgcgtgccaaacaaccacgagaggtactacggctacaccggggccttcagatgtctggtcgaaaaaggcgacgtggcattcgtgaaggaccagaccgtgctgcagaataccggcggcaagaacaccgaggattgggccaaagacctcaaagaagaggacttcgagctgctgtgtcctgacggccagagaaagagcgtggacaaggcccctgagtgctttctggccaaggctcctaatcacgccgtggtgtctcggaaggataaggcctcttgcgtgtccaagatgctgctcgaccagcaactgctgttcggcagaaacggcaatgactgcagcggcaagttctgcctgttccacagcgccaccaaggatctgctgttccgggacgatacacagtgcctggctaagctgcctgaggacaccacctacaagtcttacctgggagccgagtacatcacagccgtggccaatctgagacagtgcagcacctccaaactgctggaagcctgcaccttccacaccagctga

SEQ ID NO: 5 - protein, 110 aa, synthetic construct
  REGION 1..110: note = cINS-2-1
MALWMRLLPLLALLALWAPAPTRAFVNQHLCGSHLVEALYLVCGERGFFYTPRAKREVEDLQVRDVELAGAPGEGGLQPLALEGARQKRGIVEQCCTSICSLYQLENYCN

SEQ ID NO: 6 - protein, 708 aa, synthetic construct
  REGION 1..708: note = fINS-Alb
MAPWTRLLPLLALLSLWIPAPTRAFVNQHLCGSHLVEALYLVCGERGFFYTPRAKREAEDLQGKDAELGEAPGAGGLQPSALEAPRQKRGIVEQCCASVCSLYQLEHYCNGGGGSGGGGSGGGSEAHQSEIAHRFNDLGEEHFRGLVLVAFSQYLQQCPFEDHVKLVNEVTEFAKGCVADQSAANCEKSLHELLGDKLCTVASLRDKYGEMADCCEKKEPERNECFLQHKDDNPGFGQLVTPEADAMCTAFHENEQRFLGKYLYEIARRHPYFYAPELLYYAEEYKGVFTECCEAADKAACLTPKVDALREKVLASSAKERLKCASLQKFGERAFKAWSVARLSQKFPKAEFAEISKLVTDLAKIHKECCHGDLLECADDRADLAKYICENQDSISTKLKECCGKPVLEKSHCISEVERDELPADLPPLAVDFVEDKEVCKNYQEAKDVFLGTFLYEYSRRHPEYSVSLLLRLAKEYEATLEKCCATDDPPACYAHVFDEFKPLVEEPHNLVKTNCELFEKLGEYGFQNALLVRYTKKVPQVSTPTLVEVSRSLGKVGSKCCTHPEAERLSCAEDYLSVVLNRLCVLHEKTPVSERVTKCCTESLVNRRPCFSALQVDETYVPKEFSAETFTFHADLCTLPEAEKQIKKQSALVELLKHKPKATEEQLKTVMGDFGSFVDKCCAAEDKEACFAEEGPKLVAAAQAALA

SEQ ID NO: 7 - protein, 24 aa, synthetic construct
  REGION 1..24: note = signal peptide
MALWMRLLPLLALLALWAPAPTRA

SEQ ID NO: 8 - protein, 14 aa, synthetic construct
  REGION 1..14: note = gly/ser linker
GGGGSGGGGSGGGS

SEQ ID NO: 9 - protein, 24 aa, synthetic construct
  REGION 1..24: note = signal peptide
MAPWTRLLPLLALLSLWIPAPTRA

SEQ ID NO: 10 - protein, 86 aa, synthetic construct
  REGION 1..86: note = Canine INS - native sequence
FVNQHLCGSHLVEALYLVCGERGFFYTPKARREVEDLQVRDVELAGAPGEGGLQPLALEGALQKRGIVEQCCTSICSLYQLENYCN

SEQ ID NO: 11 - protein, 86 aa, synthetic construct
  REGION 1..86: note = Feline INS - native sequence
FVNQHLCGSHLVEALYLVCGERGFFYTPKARREAEDLQGKDAELGEAPGAGGLQPSALEAPLQKRGIVEQCCASVCSLYQLEHYCN

SEQ ID NO: 12 - protein, 86 aa, synthetic construct
  REGION 1..86: note = Canine INS (K53R, R55K, L86R )
FVNQHLCGSHLVEALYLVCGERGFFYTPRAKREVEDLQVRDVELAGAPGEGGLQPLALEGARQKRGIVEQCCTSICSLYQLENYCN

SEQ ID NO: 13 - protein, 20 aa, synthetic construct
  REGION 1..20: note = feIL2 signal peptide
MYKIQLLSCIALTLILVTNS

SEQ ID NO: 14 - protein, 15 aa, synthetic construct
  REGION 1..15: note = gly/ser linker
GGGGSGGGGSGGGGS

SEQ ID NO: 15 - protein, 86 aa, synthetic construct
  REGION 1..86: note = Feline INS (K53R, R55K, L86R)
FVNQHLCGSHLVEALYLVCGERGFFYTPRAKREAEDLQGKDAELGEAPGAGGLQPSALEAPRQKRGIVEQCCASVCSLYQLEHYCN

SEQ ID NO: 16 - protein, 584 aa, synthetic construct
  REGION 1..584: note = canine serum albumin
EAYKSEIAHRYNDLGEEHFRGLVLVAFSQYLQQCPFEDHVKLAKEVTEFAKACAAEESGANCDKSLHTLFGDKLCTVASLRDKYGDMADCCEKQEPDRNECFLAHKDDNPGFPPLVAPEPDALCAAFQDNEQLFLGKYLYEIARRHPYFYAPELLYYAQQYKGVFAECCQAADKAACLGPKIEALREKVLLSSAKERFKCASLQKFGDRAFKAWSVARLSQRFPKADFAEISKVVTDLTKVHKECCHGDLLECADDRADLAKYMCENQDSISTKLKECCDKPVLEKSQCLAEVERDELPGDLPSLAADFVEDKEVCKNYQEAKDVFLGTFLYEYSRRHPEYSVSLLLRLAKEYEATLEKCCATDDPPTCYAKVLDEFKPLVDEPQNLVKTNCELFEKLGEYGFQNALLVRYTKKAPQVSTPTLVEVSRKLGKVGTKCCKKPESERMSCADDFLSVVLNRLCVLHEKTPVSERVTKCCSESLVNRRPCFSGLEVDETYVPKEFNAETFTFHADLCTLPEAEKQVKKQTALVELLKHKPKATDEQLKTVMGDFGAFVEKCCAAENKEGCFSEEGPKLVAAAQAALV

SEQ ID NO: 17 - protein, 685 aa, synthetic construct
  REGION 1..685: note = canine transferrin
VSPEKTVRWCTVSNHEASKCSSFMENMKTVLENGPFVSCVKRTSYLECIKAIWANEADAVTLDAGLVFEAGLNPYNLKPVVAEFYGKDKQTRYYAVAVVKKSSTFNLNQLQGKKSCHTGLGRSAGWNIPMGLLYWKLPEPRESLQKAASSFFTASCVPCVDRTAFPKLCQLCVGKGTDKCACSNHEPYFGYSGAFKCLMEDAGEVAFVKHSTVLENLPNKADLDQYELLCPDNKRKPVGEYKQCYLAKVPSHAVVARSVGGKEDLIWELLNQAQEHYGKDKSKVFQLFSSTLGKDLLFKDSAEGFFRIPPKMDTWLYLGYEYVTALRNLREDVRPDTPRDECKKVKWCAVGHHEIAKCDEWSVNSEGKIECESAESTEDCIAKIAKGEADAMSLDGGYIYIAGQCGLVPVLAENYKTQGSTCSNTAEEGYLAVAVVKRLDKTISWNNLQGRKSCHTAVDRTAGWNIPMGLLYNRINHCEDKFFSQGCAPGSMRNSSLCALCIGSANVPGKECVPNNHERYYGYTGAFRCLVEKGDVAFVKDQTVLQNTGGKNTEDWAKDLKEEDFELLCPDGQRKSVDKAPECFLAKAPNHAVVSRKDKASCVSKMLLDQQLLFGRNGNDCSGKFCLFHSATKDLLFRDDTQCLAKLPEDTTYKSYLGAEYITAVANLRQCSTSKLLEACTFHTS

SEQ ID NO: 18 - protein, 584 aa, synthetic construct
  REGION 1..584: note = Feline serum albumin
EAHQSEIAHRFNDLGEEHFRGLVLVAFSQYLQQCPFEDHVKLVNEVTEFAKGCVADQSAANCEKSLHELLGDKLCTVASLRDKYGEMADCCEKKEPERNECFLQHKDDNPGFGQLVTPEADAMCTAFHENEQRFLGKYLYEIARRHPYFYAPELLYYAEEYKGVFTECCEAADKAACLTPKVDALREKVLASSAKERLKCASLQKFGERAFKAWSVARLSQKFPKAEFAEISKLVTDLAKIHKECCHGDLLECADDRADLAKYICENQDSISTKLKECCGKPVLEKSHCISEVERDELPADLPPLAVDFVEDKEVCKNYQEAKDVFLGTFLYEYSRRHPEYSVSLLLRLAKEYEATLEKCCATDDPPACYAHVFDEFKPLVEEPHNLVKTNCELFEKLGEYGFQNALLVRYTKKVPQVSTPTLVEVSRSLGKVGSKCCTHPEAERLSCAEDYLSVVLNRLCVLHEKTPVSERVTKCCTESLVNRRPCFSALQVDETYVPKEFSAETFTFHADLCTLPEAEKQIKKQSALVELLKHKPKATEEQLKTVMGDFGSFVDKCCAAEDKEACFAEEGPKLVAAAQAALA

SEQ ID NO: 19 - DNA, 2127 bp, synthetic construct
  misc_feature 1..2127: note = fINS-Alb
  source 1..2127: mol_type = other DNA
atggctccttggacgaggctgctaccgctgttggctcttctgtccctctggatacccgctcccacccgtgcatttgtcaatcagcacctctgtgggtcccaccttgttgaggccctctatctggtctgcggggagagagggttcttctacacgccccgggctaagcgcgaggcagaggacttgcagggaaaggacgctgagctcggggaagcccccggggcgggcggcctacaaccctccgccctggaggctcctcggcaaaagcgcggaattgtagagcagtgttgtgcctccgtgtgctcactctaccagctcgaacactattgtaacggggggggagggtcggggggcggcgggagcggaggcggcagcgaagctcaccagagcgaaatcgcccaccggtttaacgatttaggtgaggagcacttcagggggctggttttggttgccttcagccagtatctgcagcaatgtccattcgaggatcacgtgaagttggtgaatgaggtcactgagttcgccaaaggctgtgttgccgatcaatcagctgccaactgcgagaaatctctgcacgaacttctcggcgacaagctgtgcacggtggcctccctgcgagacaaatacggcgaaatggccgactgctgtgaaaagaaagaaccagaacgcaacgagtgtttcctccagcacaaggacgacaaccctggctttgggcaattagttaccccggaggccgacgctatgtgcaccgcattccacgaaaatgagcaacggtttctggggaagtacctctatgagatcgcacgtcgtcacccatatttttatgccccagaattactgtactatgccgaggaatataagggggtgttcaccgaatgctgcgaagccgcggacaaggctgcctgtcttactcctaaagtggacgcattacgggaaaaagtacttgctagctcagctaaagagagactgaagtgtgcttcactgcagaagtttggagaacgagctttcaaagcctggtccgtggctcggctatcgcagaaatttcccaaggctgagttcgcggagatctcaaaactggtcacagaccttgctaaaatacataaagaatgctgccacggcgacctgcttgagtgtgctgatgatcgcgccgacctggcgaaatacatatgcgagaatcaggactctatctcaacaaaactgaaagagtgttgtggtaaaccagtgttggagaaaagccattgcatttcagaggtggagcgcgacgaacttccagcggacctgccgcccttggctgtagacttcgttgaggataaggaagtgtgtaaaaactatcaggaagctaaagacgtgtttctgggcacctttctttacgagtattctaggaggcatcctgagtattctgtgagtcttctgctcaggctcgcgaaggagtacgaagcaacacttgagaaatgctgcgccacggatgatcctcccgcctgttatgcacacgtgttcgatgagttcaagcccctggttgaagagcctcacaacttggtgaagaccaattgcgaactgtttgagaaactgggggagtacggctttcagaatgctttactggtgcgctatactaagaaagttccccaagtctctacacccactctggtagaggtgagcaggtcgctgggaaaggtgggctcaaaatgctgtacccatcctgaggctgagcgtctgagttgtgccgaggattacttatccgtcgtgctgaatagactgtgcgtcctccacgagaaaaccccggtgagcgaaagggtaaccaagtgttgtaccgaatcactggttaataggaggccttgcttctccgctttgcaggtggacgaaacctatgtgcctaaggagttctccgccgaaacatttaccttccacgccgacctctgcactttacccgaagcagagaagcagataaagaagcagtccgctctagtcgaattgctgaaacataagccaaaagcaacagaagagcagcttaaaaccgtgatgggcgacttcggttccttcgttgacaagtgctgtgccgccgaggacaaagaagcgtgcttcgccgaggaaggtcctaagctggtcgcggcggctcaggcggcattggcttga

SEQ ID NO: 20 - protein, 10 aa, synthetic construct
  REGION 1..10: note = gly/ser linker
GGGGSGGGGS

SEQ ID NO: 21 - protein, 20 aa, synthetic construct
  REGION 1..20: note = caIL2 signal peptide
MYKMQLLSCIALTLVLVANS